SEQUENCE LISTING (5 sequences)

SEQ ID NO: 1 - RNA, 75 nt, Artificial sequence
  Description of the artificial sequence  RNA primer
ggccgugaucguauagdggudaguacucugcguuguggccgcagcaacccgguucgaauccgagucacggcacca

SEQ ID NO: 2 - RNA, 108 nt, Artificial sequence
  Description of the artificial sequence  RNA template
nnnnncucugguaacuagagaucccucagaccauuuuaguuagugugccacaacgcuagcaauggugccgugacucggaucugaaagcgaaagggaaaccagagnnnn

SEQ ID NO: 3 - RNA, 76 nt, Artificial sequence
  Description of the artificial sequence  RNA primer
gccucguuagcgcaguagguagcgcgucagucucauaaucugaaggucgugaguucgauccucacacggggcacca

SEQ ID NO: 4 - RNA, 110 nt, Artificial sequence
  Description of the artificial sequence  RNA template
nnnnncucugguaacuagagaucccucagacccuuuuaguucaguguugugagacuguaacaguggugccccgugugaggcaugaaagcgaaagggaaaccagaggnnnn

SEQ ID NO: 5 - DNA, 18 bp, Artificial sequence
  Description of the artificial sequence  primer
atccgagtcacggcacca

(19) United States
(12) Patent Application Publication
(10) Pub. No.: US 2006/0216693 A1
(43) Pub. Date: Sep. 28, 2006

(54) Method of screening compounds which inhibit the initiation of the retrotranscription of the rna of virus hiv-1 and means for implementing same

(75) Inventors: Fabienne Brule, Saint Denis En Val (FR); Catherine Isel, Strasbourg (FR); Roland Marquet, Illkirch (FR); Mickäel Rigourd, Bordeaux (FR)

Correspondence Address: FULBRIGHT & JAWORSKI L.L.P., 600 CONGRESS AVE., SUITE 2400, AUSTIN, TX, 78701, US

(73) Assignees: Centre National de la Recherche Scientifique (CNRS, Paris Cedex 16 (FR); Universite Strasbourg 1 Louis Pasteur, Strasbourg (FR)

(21) Appl. No.: 10/546,353

(22) PCT Filed: Feb. 19, 2004
(86) PCT No.: PCT/FR04/50068

§ 371 Date: Feb. 13, 2006

(30) Foreign Application Priority Data
Feb. 20, 2003 (FR) 03/50029

(51) Int. Cl. C12Q 1/70; C07H 21/02

(52) U.S. Cl. 435/5; 536/23.72

(57) ABSTRACT

The present invention relates to methods for screening compounds that can inhibit the early stage of an infection by the HIV-1 virus, that is to say the initiation step of viral genome reverse transcription, before integrating viral sequences into the cellular genome of the infected host organism.

### FIELD OF THE INVENTION

The present invention relates to the field of preventive or curative treatment for human diseases induced by an infection by the human immunodeficiency virus type 1 (HIV-1).

It relates to methods for selecting compounds that can prevent an early step of a HIV-1 virus-induced infection from proceeding in the human body, that is to say the initiation step of the viral genome reverse transcription before the viral sequences become integrated into the cellular genome of the infected host organism.

### PRIOR ART

Twenty years after the first diagnosis of acquired immune deficiency syndrome has been established, AIDS has become the most devastating disease in the world with about 42 million people being infected by the HIV virus to date.

For 2002 only, more than 3 million people have contracted the virus, whereas 3 million people died from the disease. Since the discovery in 1983 of the most significant AIDS pathogenic agent, the HIV-1 virus, very substantial efforts have been made to understand the virus mechanism of action and to develop preventive means and to search for curative means to fight against the disease.

Today, anti-HIV compounds used in therapy essentially target two enzymes that play a crucial role in the virus replicative cycle, i.e. viral protease and reverse transcriptase, respectively. Until now, seven protease inhibiting compounds have been traditionally used in therapy and other protease directed molecules are being clinically tested. Moreover, ten viral reverse transcriptase inhibiting compounds are nowadays commonly used in therapy, i.e. seven nucleoside inhibitors and three non nucleoside inhibitors, respectively, other reverse transcriptase inhibitors being at the present time clinically tested.

Preliminary assays have also been conducted with active inhibitors at different stages of HIV infection, especially with virus-cell fusion inhibitors, zinc finger inhibitors of nucleocapside protein and integrase inhibitors.

Nowadays, the treatment of HIV-1 virus-induced infections occurs as a multi-drug therapy or HAART (Highly Active Anti-Retroviral Therapy). In the case of multi-drug therapies, a protease inhibitor and two reverse transcriptase inhibitors are generally combined. These inhibitor mixtures extremely differ the disease spreading and improve the everyday life of the patients for a long period of time.

Nevertheless, the current multi-drug therapies are not able to fully eradicate the virus from the organism of infected people. One of the main difficulties encountered is the rapid growth of resistant virus strains that multiply resistance mutations in protease-encoding genes and/or reverse transcriptase-encoding genes. Protease inhibitor resistance mainly appears as mutations developing in the enzyme active site. Resistance to reverse transcriptase analogue non nucleoside inhibitors results from mutations occurring in the hydrophobic cavity that fixes these inhibitors. Reverse transcriptase nucleoside inhibitor resistance results from localized mutations that occur in the fingers and in the palm region of the enzyme.

Therefore, there is a need in the state of the art for improving the efficiency of current anti-AIDS treatments. It would be especially highly important to complete the whole range of anti-viral compounds so as to develop new compositions to be used particularly in multi-therapy and which could maintain a good anti-viral efficiency as mutant viral strains appear that are resistant to one or more anti-viral compounds currently used.

The therapeutic efficiency of preventive or curative pharmaceutical compositions against AIDS could especially be improved by using active compounds directed against targets different from the only HIV protease and reverse transcriptase.

### SUMMARY OF THE INVENTION

The present invention provides methods for screening useful compounds in the context of an anti-AIDS therapy, and more specifically, active compounds to inhibit or to prevent the early stage in a host organism of a HIV-1 virus-induced infection, i.e. the reverse transcription stage.

More specifically, the present invention relates to methods for screening compounds that are able to inhibit the reverse transcription process first stage of viral genome RNA, in other words the reverse transcription initiation step.

The present invention provides means for conducting such screening methods in the form of RNA/RNA complexes between a RNA primer and a RNA template, wherein RNA primer does not comprise any post-transcription modification of its constitutive bases.

The present invention also relates to kits for screening compounds inhibiting the initiation of HIV-1 virus RNA reverse transcription comprising a RNA primer/RNA template complex such as defined hereinabove.

### DESCRIPTION OF THE FIGURES

FIG. 1: Secondary Structure Model of the tARN^{Met}/vRNA [Met-AC] Complex

FIG. 1A: sequences corresponding to nucleotides 131 to 205 of Hxb2 isolate from viral genome RNA of wild-type and adapted HIV-1. Adapted vRNA [Met-AC] contains a PBS sequence (Primer Binding Site) complementary to the 18 nucleotides of the tRNA^{Met} 3′ end, a sequence upstream of the PBS complementary to the tRNA^{Met} anticodon loop and additional mutations (encircled) that appeared after prolonged cell culture.

FIG. 1B: Complex Secondary Structure

vRNA [Met-AC] is in black, tRNAMet is in white on a black background. The interaction between tRNA^{Met} anticodon loop and vRNA complementary region, upstream of the PBS, corresponds to helix 6C and that of the tRNA^{Met} 3′ end together with the PBS forms helix 7F. The other intermolecular interactions correspond to helices 3E and 5D. Encircled nucleotides correspond to mutations that appeared after a prolonged cell culture.

FIG. 2: Secondary Structure Model of the tRNA^{His}/vRNA [His-AC-GAC] Complex

FIG. 2A: sequences corresponding to nucleotides 131 to 205 of Hxb2 isolate of viral genome RNA of wild-type HIV-1 (highest line) and adapted HIV-1 (lowest line). Adapted vRNA [His-AC-GAC] contains a PBS sequence complementary to the 18 nucleotides of the tRNA^{His} 3′ end, a sequence CCACAA upstream of the PBS complementary to the tRNA^{His} anticodon loop and additional mutations (encircled) that appeared after prolonged cell culture.

FIG. 2B: Complex Secondary Structure

vRNA [His-AC-GAC] is in black, tRNA^{His} is in white on a black background. The interaction between tRNA^{His} anticodon loop and vRNA complementary region, upstream of the PBS, corresponds to helix 6C and that of the tRNA^{His} 3′ end together with the PBS forms helix 7F. The encircled nucleotides correspond to mutations that appeared after a prolonged cell culture.

FIG. 3: S1 Nuclease Mapping of Single-Strand Regions for Free tRNA^{His} (the 3 Lanes on the Left) or Hybridized to vRNA [His-AC-GAC] (the Next 3 Lanes)

0 lanes are incubation controls with no S1 nuclease whereas 7.5 and 15 lanes correspond to 7.5 and 15-minute incubations, respectively, with 200 U S1 nuclease.

T1 and L lanes (far right of the figure) correspond respectively to T1 Rnase sequencing with a scale resulting from alkaline hydrolysis.

FIG. 4: Minus Strand “Strong-Stop” DNA Synthesis from Natural or Synthetic tRNA^{His}

Template/primer complex (10 nM) was incubated with HIV-1 reverse transcriptase (RT) (27 nM). Initiation of the reaction is made by adding 1 μl [α-³²P] dCTP (3000 Ci/mmol), 15 μM dCTP and 150 μM of each of the three other dNTP. Reaction times are 5, 10, 15, 20, 25 and 30 minutes.

FIG. 5: tRNA^{His} and ODN_{His} extension cinetics with RNAv [His-AC-GAC]

FIG. 5A: (−) strand strong-stop DNA synthesis from tRNA^{His} with or without any poly(rA)/oligo(dT)₁₈ trap that only permits one polymerization cycle.

Template/primer complex (2 nM) was incubated with HIV-1 reverse transcriptase (RT) (20 nM) and initiation of the polymerization reaction is made by adding 50 μM of each dNTP, with or without 1,66 μM poly(rA)/oligo(dT)₁₈. Reaction was stopped at various times ranging from 15 seconds to 30 minutes.

FIG. 5B: (−) strand strong-stop DNA synthesis from ODN^{His} with or without any poly(rA)/oligo(dT)₁₈ trap that only permits one polymerization cycle.

ODN^{His} has the following sequence such as set forth in SEQ ID NO:5:

5′-ATCCGAGTCACGGCACCA-3′

Template/primer complex (10 nM) was incubated with HIV-1 reverse transcriptase (RT) (30 nM) and initiation of the polymerization reaction is made by adding 50 μM of each dNTP with or without 1,66 μM poly(rA)/oligo(dT)₁₈. Reaction was stopped at various times ranging from 15 seconds to 30 minutes.

FIG. 6: (−) strand strong-stop DNA synthesis from biotinylated vRNA complex [His-AC-GAC]/tRNA^{His} with AZTTP increasing concentrations (from 0.1 to 5 μM) (FIG. 6A) and with d4TTP increasing concentrations (from 0.1 to 5 μM) (FIG. 6B).

10 nM vRNA [His-AC-GAC]/tRNA^{His} preformed complex are transferred into the wells of a microplate of “Flashplate” type (NEN) and incubated with 5 μM of each dNTP (dATP, dCTP, dGTP and dTTP/dTTP[³H], 30 nM HIV-1 RT and without or with nucleoside inhibitors. The reaction proceeds at 37° C. for 5, 15 or 30 minutes before being stopped by the addition of 25 mM EDTA. Counting of the surface-bound radioactivity for each microplate well is performed with a MicroBeta counter (Mallac-Perkin-Elmer) according to a Scintillation Proximity Assay (SPA).

The X-axis on the graph corresponds to AZTTP concentrations in μMol/litre; the Y-axis corresponds to the scintillation signal in cpm.

FIG. 7: (−) Synthesis of a Product Corresponding to Retrotranscription Initiation from from Biotinylated vRNA complex [His-AC-GAC]/tRNA^{His}

30 nM of preformed template/primer complex are transferred into the wells of a microplate of “Flashplate” type (NEN) and incubated with of dGTP, dCTP, dTTP/dTTP[³H] (5 μM final each) ddA (20 μl), 90 nM HIV-1 RT and without or with AZTTP. The reaction proceeds at 37° C. for 20 minutes before being stopped by the addition of 25 mM EDTA. Synthesis of a product corresponding to the addition of 6 nucleotides to the primer is detected with a MicroBeta counter (Mallac-Perkin-Elmer) according to a Scintillation Proximity Assay (SPA).

The signal attenuation is detected with 0.1 and 2.5 μl of AZTTP. The numbers in bold and italic on the top of histograms corresponds to the percent of residual signal compared to the control without inhibitor.

The X-axis on the graph corresponds to AZTTP concentrations in μMol/litre; the Y-axis corresponds to the scintillation signal in cpm.

### DETAILED DESCRIPTION OF THE INVENTION

The present invention provides methods for screening compounds inhibiting the initiation of HIV-1 virus RNA reverse transcription. The development of screening methods according to the invention was made possible thanks to the preparation of novel RNA/RNA complexes that can mimic the reverse transcription natural initiation complex which is formed once the HIV-1 virus has come into the cell.

Reverse transcription is a key step of retrovirus replication cycle, for HIV-type virus as well, especially HIV-1. The first step of the reverse transcription cycle consisting in synthesizing a DNA strand called minus (−) strand strong-stop DNA.

Synthesizing a (−) strand strong-stop DNA consists in elongating isoacceptor 3 of tRNA specific to cellular lysine (tRNA₃^{Lys}), from a template comprising the viral genome RNA. The 18 nucleotides localized on tRNA₃^{Lys} 3′ end are strictly complementary to the primer binding sequence (PBS) of HIV-1 genome RNA localized close to viral genome 5′ end. Based on the reverse transcription initiation RNA/RNA complex formed between tRNA₃^{Lys} and viral genome RNA, HIV-1 reverse transcriptase uses 3′-OH fragment of tRNA primer hybridized to viral genome RNA to initiate the (−) strand strong-stop DNA synthesis.

According to the state of the art, it has been demonstrated that (viral RNA/tRNA₃^{Lys}) complex for HIV-1 reverse transcription initiation has specific structural and functional characteristics. It has been shown by means of chemical mapping and enzyme solution mapping that interaction between tRNA₃^{Lys} and viral genome RNA is not limited to the 18 nucleotides comprising the primer binding sequence (PBS) but also presents various intra- and intermolecular structural rearrangements to result in a compact and very complex structure (ISEL and al., 1995; ISEL and al., 1996).

Amongst the intermolecular interactions, pairing between the viral RNA A-rich loop and the tRNA₃^{Lys} anticodon loop plays a crucial role in stabilizing the structure of the reverse transcription initiation complex. It has been especially demonstrated that stabilizing additional interactions strongly depends on the presence of bases that have been modified by intracellular post-transcriptional modification of natural transfer RNA (Isel and al., 1996; Isel and al., 1993; Skripkin and al., 1996).

A functional specificity responds to the structural specificity of the HIV-1 reverse transcription initiation complex. Kinetics studies have demonstrated that the DNA (−) strand strong-stop synthesis comprises two steps: 1) an initiation step, specific to tRNA₃^{Lys}, corresponding to the addition of the six first nucleotides at the natural primer 3′ end, followed by 2) a non specific elongation step, which can be mimiced by a 18 nucleotide-long DNA primer strictly complementary to the PBS. (Isel and al., 1996; Lanchy and al., 1996).

It has also been demonstrated that the reverse transcription initiation step is distributive; it is characterized by a slow nucleotide incorporation rate and by a high reverse transcriptase dissociation rate of the template/primer complex (viral RNA/tRNA₃^{Lys}) (Lanchy and al., 1996). On the other hand, the elongation step, corresponding to the addition of the subsequent nucleotides from a primer that has become a non specific oligodeoxyribonucleotide primer, is processive; it is characterized by a high nucleotide incorporation rate and by a slow reverse transcriptase dissociation rate (Lanchy and al., 1996; Lanchy and al., 1998).

Isel and al. also observe (1996) that nucleosides that have been modified by intracellular post-transcriptional modifications in natural transfer RNA are necessary to prepare an efficient reverse transcription initiation step by increasing HIV-1 reverse transcriptase affinity for the tRNA₃^{Lys}/RNA binary complex (Isel and al., 1996; Lanchy and al., 1996; Isel and al., 1993; Isel and al., 1999). It has also been demonstrated that additional template/primer interactions make the transition easier from the initiation step to the elongation step.

Experiment results obtained by Lanchy and al. (1996) as well show that intracellular post-transcriptional modifications of the transfer RNA are required to form a stable and active initiation complex. The obtained results show that extension rates of the primer prepared with the natural purified tRNA₃^{Lys} are about 32 to 35 times higher than the extension rate of the primer prepared with the tRNA₃^{Lys} transcripted in vitro, which one does not comprise any post-transcriptional modification and therefore does not have any modified base. The authors conclude that tRNA free from any modified base is not specifically recognized as a primer by the HIV-1 reverse transcriptase. To their mind, bases that have been modified by a post-transcriptional modification of the cellular natural tRNA are involved in the formation of the initiation complex and are likely to anchor HIV-1 reverse transcriptase into the tRNA primer/viral RNA template complex, thus explaining reverse transcriptase loss of specificity observed with a synthetic tRNA which does not comprise any modified base.

It has thus been admitted that post-transcriptional modifications of lysine transfer RNA stabilize primer/template interactions within the reverse transcription initiation complex (Isel and al., 1993). A stable primer/template complex cannot be formed if transfer RNA tRNA₃^{Lys} without any base modified by post-transcriptional modifications is used. The authors put the emphasis on the necessity to use a natural purified transfer RNA to form a stable and active reverse transcription initiation complex.

Moreover, Isel and al. (1996) observe that nucleosides that have been modified by intracellular post-transcriptional modifications of the natural tRNA are required to prepare an efficient reverse transcription initiation step and for a good transition from the initiation step to the elongation step.

The functional importance of the interaction between the tRNA₃^{Lys} anticodon loop and the A-rich loop has been demonstrated in vivo. Mutant virus that do not possess this complementarity anymore suffer indeed from replication defaults and progressively restore the A loop in prolonged culture (Huang and al., 1996, Liang and al., 1997). HIV-1 virus variants, the PBS of which has undergone a mutation so as to be compatible with tRNA^{His} or tRNA^{Met} stably use these tRNA, with the proviso that the A loop has simultaneously undergone a mutation so as to be complementary to the anticodon of these tRNA (Kang and al., 1997, Wakefield and al., 1998). Analysing the mutants that stably use tRNA^{His} revealed that they comprise, in addition to mutations in the PBS and in the A loop (Zhang and al., 1996), three secondary mutations in U5, upstream of the PBS, formed in prolonged cultures and involved in maintaining tRNA^{His} as a primer (Zhang and al., 1998).

Now, we have surprisingly demonstrated according to the invention that a RNA primer/viral RNA template complex that mimics the initiation complex of HIV-1 virus reverse transcription could be obtained between a viral RNA template and a RNA primer free from any post-transcriptional modification.

So the applicant could prepare a RNA primer/viral RNA template complex between a histidine transfer RNA (tRNA^{His}) prepared by means of an in vitro transcription and a viral RNA the sequence of which has been adapted so as to be complementary to tRNA^{His} at the codon sequence and at the primer binding sequence (PBS), tRNA^{His} having no base comprising any chemical modification similar to a natural post-transcriptional modification.

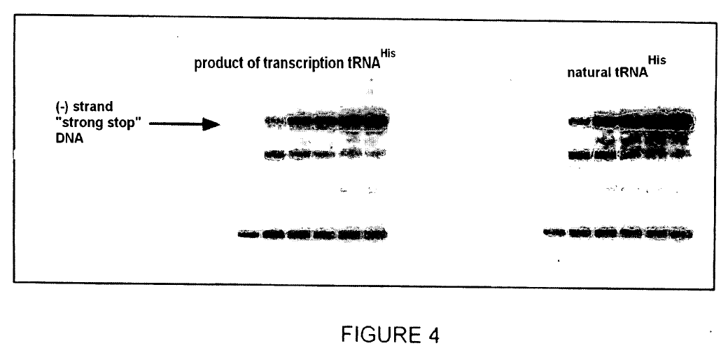

The hereinabove RNA primer/viral RNA template complex is stable and makes it possible to efficiently elongate RNA primer (tRNA^{His} without any modified base) according to the initiation/elongation model as previously defined and illustrated in FIG. 4.

The applicant also describes a RNA primer/RNA template complex with a methionine transfer RNA (tRNA^{Met}) obtained by in vitro transcription and with a viral RNA the sequence of which has been adapted so as to be complementary to tRNA^{Met} at the codon sequence and at the primer binding sequence (PBS)

Experiment results that are given in the examples surprisingly reveal that certain tRNA primer obtained by simply transcripting in vitro DNA encoding the latter, the bases of which having therefore not undergone any post-transcriptional modification, can form a RNA primer/RNA template complex with a viral RNA template, the sequence of which has been adapted (i) at viral RNA sequence that is complementary to the primer anticodon sequence (thereinafter also called “codon sequence”) and (ii) at the primer binding sequence (PBS). It has also been demonstrated that with a reverse transcriptase, the RNA primer/viral RNA template complex is biologically active and can mimic the initiation step, then the elongation step in the HIV-1 viral genome reverse transcription.

According to the present invention, RNA primer/viral RNA template complexes can now be produced in large amounts that mimic the HIV-1 reverse transcription initiation complex. Indeed synthesizing a RNA primer with no post-transcriptional modification can be conducted economically and in large amounts, for instance by simply transcripting in vitro a DNA encoding said RNA primer. Indeed, as already stated hereabove, stable and active HIV-1 reverse transcription initiation complexes, according to the state of the art, were formed from a natural transfer RNA, obtained according to long, complicated and very expensive purification methods, especially from beefs or chicken's liver.

Thanks to the present invention, the accessibility of large amounts of RNA primer/viral RNA template complexes without any post-transcriptional modification and mimicking the activity of the natural initiation complex of the HIV-1 reverse transcription makes it finally possible to use these RNA primer/viral RNA template complexes for selecting compounds inhibiting the HIV-1 natural initiation complex activity on a large scale.

It has been indeed demonstrated according to the invention that using a RNA primer/viral RNA template complex wherein RNA primer is an in vitro transcription product with no post-transcriptional modification, when in the presence of a HIV-1 reverse transcriptase, makes it possible to detect the inhibition activity of a candidate compound such as AZTTP on the initiation step of HIV-1 viral genome RNA reverse transcription.

So for the first time, the present invention provides those skilled in the art with a method for screening compounds inhibiting the initiation of HIV-1 reverse transcription. Therefore, the reverse transcription initiation complex now represents thanks to the present invention a novel therapeutic target, the blockage or the inhibition of which offers an additional therapeutic anti-HIV-1 strategy that can easily fit into a HAART (Highly Active Anti-Retroviral Therapy) multi-drugs therapy. Introducing compounds inhibiting the initiation step of viral reverse transcription in the context of multi-drugs regimens should drastically reduce the risk of developing resistant HIV-1 strains. There is every likelihood that using compounds selected according to this screening method and able to inhibit the HIV-1 reverse transcription initiation step will reduce the risk of developing resistant variant viral strains, since one of the components of the involved intracellular target, that is to say the natural transfer RNA, cannot undergo any mutation without simultaneously seriously damaging or even fully blocking the protein synthesis within the cell, which also indirectly represents a very strong screening pressure limiting variation or mutation risks on the viral genome RNA 5′ end, which in many regions has to be complementary to the tRNA primer sequence.

RNA/RNA Complexes According to the Invention that Mimic the HIV-1 Virus RNA Reverse Transcription Initiation Complex.

A RNA primer/RNA template complex according to the invention that mimic the HIV-1 virus RNA reverse transcription initiation complex is characterized in that it comprises a RNA primer/RNA template complex formed between:

(i) a RNA primer comprising from the 5′ end to the 3′ end:
an intramolecular pairing sequence (109);
a single-strand sequence (111);
a stem-loop forming sequence (112);
a single-strand sequence (113);
an intermolecular pairing sequence (101) called anticodon sequence;
an intermolecular pairing sequence (103);
an intermolecular pairing sequence (105);
an intramolecular pairing sequence (110);
a single-strand sequence (114);
an intermolecular pairing sequence (107) used as a primer; and

(ii) a RNA template comprising from the 5′ end to the 3′ end:
an intramolecular pairing sequence (115);
an intramolecular pairing sequence (116);
an intermolecular pairing sequence (106);
an intramolecular stem-loop forming sequence (117);
an intramolecular pairing sequence (104);
an intramolecular pairing sequence (102) called codon sequence;
an intramolecular pairing sequence (118);
a single-strand sequence (119);
an intermolecular pairing sequence (108);
a sequence (120) forming the intramolecular stem-loop (8);
a single-strand sequence (121);
an intramolecular pairing sequence (122);

wherein the RNA primer consists in:
(a) either the in vitro transcription product of a nucleic acid encoding said RNA primer, upstream which a transcription promoter site is localized, such as T7 promoter;
(b) or the chemical synthesis or chemical hemisynthesis product of said RNA primer;

being specified that:
the paired sequences (101) and (102) form a (6C) helix;
the paired sequences (107) and (108) form a (7F) helix;
the paired sequences (109) and (110) form a (A) helix;
the paired sequences (107) and (108) form a (7F) helix;
the sequence (112) forms a stem-loop (B);
the paired sequences (115) and (122) form a helix (1);
the paired sequences (116) and (118) form a helix (2);
the sequence (117) forms a stem-loop (4);
the sequence (120) forms a stem-loop (8).

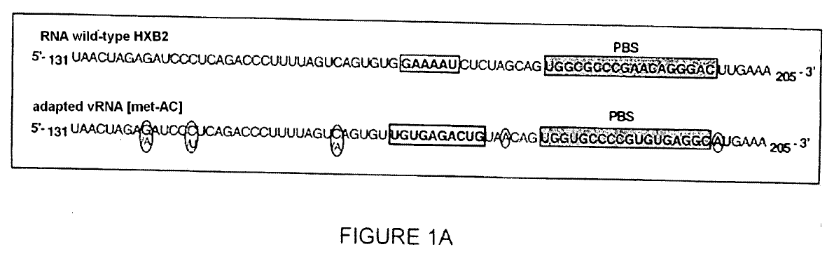

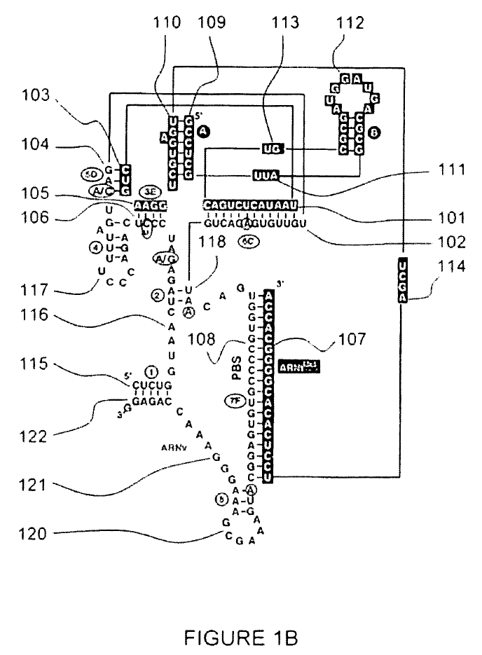

Moreover, for a first RNA primer/RNA template complex family according to the invention, (i) the paired sequences (103) and (104) form a helix (5D) and (ii) the paired sequences (105) and (106) form a helix (3E). That is the case of the RNA primer/RNA template complex shown in FIG. 1B.

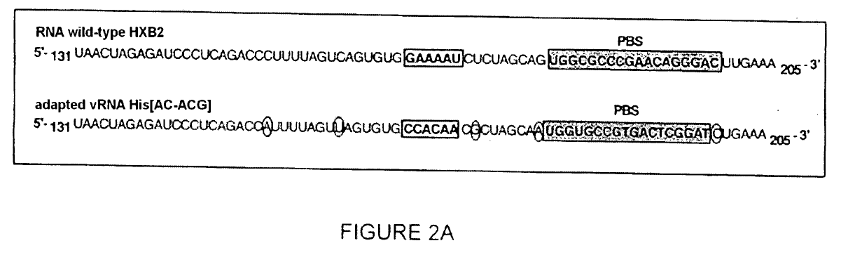

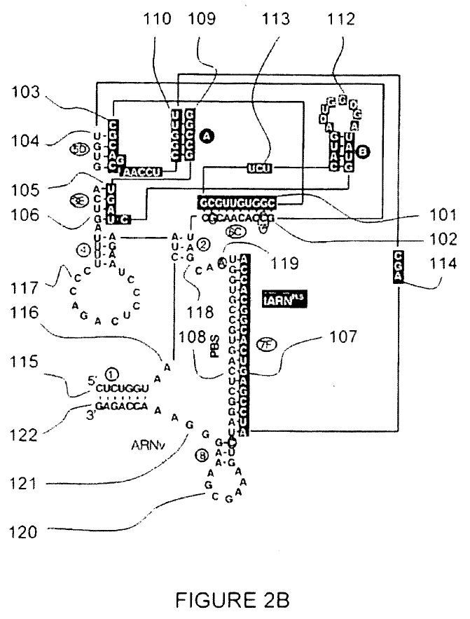

Moreover, for a second family of RNA primer/RNA template complexes according to the above definition given for the above reverse transcription initiation complex, the structure differs from the one described for the first sheet hereinabove for the helices 3E and 5D and the stem-loop 4. For example, FIG. 2 shows this difference, that results nevertheless in a structure similar to the previous one et responds to the initiation complex rules such as defined by the inventors. That is the case of the RNA primer/RNA template complex shown in FIG. 2B.

The above RNA primer/RNA template complex is also characterized in that it mimics the initiation step of HIV-1 viral genome RNA reverse transcription according to a kinetics that is characteristic for HIV-1 reverse transcription initiation. Functional characteristics of a RNA primer/RNA template complex according to the invention as regards reverse transcription initiation are described in example 2.

These characteristics comprise a distributive incorporation of the six first nucleotides at RNA primer 3′ end.

According to the invention, it is demonstrated that with a RNA primer such as defined hereabove there are marked “pauses” during the polymerization of the first 11 nucleotides, “pauses” that are rarefying with time. This means that reverse transcriptase during the polymerization of these first 11 nucleotides is not processive and tends to separate easily after a nucleotide has been added. This is even more fully explained by the fact that with a trap (polyrA/oligodT) the entire reverse transcriptase is “trapped in the trap” and polymerization cannot occur.

On the other hand, with a oligonucleotide primer that can mimic the elongation, the “pauses” are not so numerous.

It has to be emphasized that with a trap, elongation polymerization will occur in spite of everything and a substantial amount of final product will be detected, that means well that reverse transcriptase is very processive and can polymerize about 200 nucleotides in this system without coming off its substrate.

According another interesting characteristic, a RNA primer/RNA template complex according to the invention must also satisfy following kinetics criteria: DNA synthesis from the RNA primer proceeds in two phases: a distributive initiation phase and a processive elongation phase, kinetics thereof being easily measured such as described by Lanchy and al. (EMBO; 1996).

Distribution between the two kinetics, respectively the distributive and the processive ones, may be demonstrated with a polyrA/oligodT trap, as described in example 2.

According to a preferred embodiment, the above RNA primer/RNA template complex comprises, respectively:

(i) a RNA primer selected from:
(a) the in vitro transcription product of a nucleic acid, DNA, or RNA, encoding a transfer RNA;
(b) a RNA primer produced by chemical synthesis or chemical hemisynthesis, the nucleotide sequence of which corresponds to that of a transfer RNA naturally occurring in human cells;

and

(iii) a RNA template consisting in a HIV-1 virus genome RNA-derivatized RNA, if necessary at least adapted in the codon sequence (102) and in the primer binding sequence PBS (108), so that the primer binding sequence (108) is strictly complementary to the used RNA primer sequence (107) and the anti-codon sequence (101) is strictly complementary to the codon sequence (102). Base complementarity of sequence (108) to sequence (107) corresponding bases makes sure that a stable and functionally active RNA primer/RNA template complex is formed to mimic HIV-1 virus reverse transcription initiation with a HIV-1 reverse transcriptase.

Other interesting characteristics of a RNA primer according to the present invention are as follows:
sequence (109) preferably is a 8 nucleotide-long sequence;
sequence (111) preferably is a 2 nucleotide-long sequence;
sequence (112) preferably is a 16 nucleotide-long sequence;
sequence (113) preferably is a 4 nucleotide-long sequence;
sequence (101) preferably is a 11 nucleotide-long sequence;
sequence (103) preferably is a 3 nucleotide-long sequence;
sequence (105) preferably is a 4 nucleotide-long sequence;
sequence (110) preferably is a 6 nucleotide-long sequence;
sequence (114) preferably is a 4 nucleotide-long sequence; and
sequence (107) preferably is a 18 nucleotide-long sequence.

Other interesting characteristics of a RNA template according to the present invention are as follows:
sequence (115) preferably is a 5 nucleotide-long sequence;
sequence (116) preferably is a 12 nucleotide-long sequence;
sequence (106) preferably is a 5 nucleotide-long sequence;
sequence (117) preferably is a 10 nucleotide-long sequence;
sequence (104) preferably is a 6 nucleotide-long sequence;
sequence (102) preferably is a 13 nucleotide-long sequence;
sequence (118) preferably is a 3 nucleotide-long sequence;
sequence (119) preferably is a 3 nucleotide-long sequence;
sequence (108) preferably is a 18 nucleotide-long sequence;
sequence (120) preferably is a 13 nucleotide-long sequence;
sequence (121) preferably is a 6 nucleotide-long sequence; and
sequence (122) preferably is a 5 nucleotide-long sequence.

FIGS. 1 and 2 show different embodiments of a RNA primer/RNA template complex according to the invention that mimics the initiation complex of the HIV-1 virus genome RNA reverse transcription. For each of the thus illustrated embodiments, RNA template sequences, more specifically the codon sequence (102) and the primer binding sequence (108) have been adapted so as to optimise base pairing respectively with the “anticodon sequence” (101) and primer (107) of RNA primer, so as to form helices (6C) and (7F), respectively.

FIG. 1 represents the RNA primer/RNA template complex that has been formed between RNA primer comprising a sequence as set fort in SEQ ID NO3 being an in vitro transcription product and RNA template comprising a sequence as set fort in SEQ ID NO4.

FIG. 2 represents the RNA the primer/viral RNA template complex that has been formed between RNA primer comprising a sequence as set fort in SEQ ID NO1 being an in vitro transcription product and RNA template of sequence SEQ ID NO2.

For RNA primer/RNA template complexes such as defined hereabove, it has been demonstrated according to the invention by means of mapping experiments after S1 nuclease action that RNA primer formed by in vitro transcription of DNA encoding it and which thus does not have any post-transcriptional modified base surprisingly forms a stable complex with the corresponding RNA template. It has thus been demonstrated that the thus obtained stable RNA primer/RNA template complex mimics the structural characteristics of the reverse transcription initiation complex that characterize the complex formed between natural tRNA₃^{Lys} and the viral genome RNA region corresponding to the tRNA₃^{Lys} natural primer. In particular, it has been demonstrated that RNA primer/RNA template interactions comprise the pairing between the codon sequence (102) and the anticodon sequence (101) and not only the interaction between the primer sequence (107) and the primer binding sequence (108).

A RNA primer/RNA template complex such as previously defined is the object of the present invention.

A first preferred RNA primer/RNA template complex according to the invention is the one formed between:
the RNA primer comprising a sequence as set forth in SEQ ID NO1, with no post-transcriptional modified base; and
a RNA template comprising a sequence as set forth in SEQ ID NO2.

A second preferred RNA primer/RNA template complex according to the invention is the one formed between:
the RNA primer comprising a sequence as set forth in SEQ ID NO3, with no post-transcriptional modified base; and
a RNA template comprising a sequence as set forth in SEQ ID NO4.

A RNA template comprising a sequence as set fort in SEQ ID NO2 or in SEQ ID NO 4 may be up to 10000 nucleotide-long. Such a RNA template may for instance be formed according to a method comprising the following steps of:

a) amplification, for ex. by a polymerase chain reaction (PCR), of the interesting DNA sequence of a clone containing a complementary DNA insert derivatized from HIV-1 virus genome RNA, said cDNA insert containing the sequence corresponding to viral genome 5′ end. The amplification is advantageously conducted by means of a couple of nucleotide primers containing appropriate sequences, a first primer hybridizing with a cDNA insert sequence localized downstream (on the 3′ end side) of the primer binding sequence (PBS) and a second primer hybridizing with a predefined sequence of the cDNA insert localized upstream (on the 5′ end side) of the primer binding sequence (PBS), respectively. In the most preferred way, respective first and second primer sequences are selected so as to produce an amplified DNA the sequence of which comprises at least RNA template sequences (115) and (122) that are complementary to each other and the complementary bases of which are paired two by two. As an illustration, the cDNA insert used may be the cDNA insert contained in the infectious molecular clone pHXB2(His-AC-AGC) described by Li and al. (1997). According to a preferred embodiment of the step a) of the process, the first nucleotide primer used comprises the promoter sequence which will be localized within the amplified DNA upstream of the 5′ end of the DNC encoding RNA primer, said promoter being functional so as to effect the in vitro transcription of the DNA encoding the RNA template. The promoter may be inter alia promoter T7, as will be described in example 1.

b) if needed, adaptation of the RNA template sequence formed in step a) may be effected by a site-directed mutagenesis method for instance by means of primers bearing the desired mutation, used in an PCR amplification. This enables a RNA template to be obtained, the sequence of which is adapted so as to ensure its perfect base complementarity to the RNA primer sequence used in the nucleotide pairing regions such as previously defined between the two sequences within the RNA primer/RNA template complex.

c) in vitro transcription of the DNA encoding the RNA template, for example as described in example 1.

A particular embodiment of how to prepare a RNA primer hereabove is illustrated in example 1.

The RNA primer/RNA template complex is preferably prepared from a RNA primer and a RNA template, respectively, according to the method described by Isel and al. (1993).

Method for Screening in vitro Compounds Inhibiting the Initiation of HIV-1 Reverse Transcription

RNA primer/RNA template complex preparation such as defined hereabove enabled the applicant to develop a method for screening in vitro compounds inhibiting the initiation of HIV-1 virus RNA reverse transcription. The availability of the above RNA primer/RNA template complexes in very large amounts prepared using a RNA primer with no natural post-transcriptional modification of its bases made it possible to develop a screening method fully conducted in vitro, which can therefore by put into practice rapidly, on a very large scale, economically and very easily since no cell culture is required.

It is thus an object of the present invention to provide a method for screening in vitro compounds inhibiting the initiation of HIV-1 virus RNA reverse transcription, characterized in that it comprises the following steps consisting in:

a) contacting in a sample
(i) a RNA primer/RNA template complex such as previously defined; with
(ii) a HIV-1 reverse transcriptase enzyme; and
(iii) an appropriate dNTPs/ddNTP mixture;
(iv) in presence of a candidate compound to be tested;

b) measuring the reverse transcription initiation activity in said sample;

c) comparing the measured activity in step b) to the reverse transcription initiation activity measured in a control sample for which step a) is conducted without said candidate compound.

The above screening method may further comprise the following step:

d) selecting the candidate compound for which reverse transcription initiation activity measured in step b) is lower than that measured for the control sample;

e) calculating the candidate compound inhibition activity selected in step d).

As previously stated, one of the main characteristics of the RNA primer/RNA template complex used in the hereabove method for screening inhibitors of the initiation of HIV-1 virus RNA reverse transcription is that the complex RNA primer does not contain any post-transcriptional modification.

According to a first aspect, the RNA primer in the RNA primer/RNA template complex corresponds to the transcription product in vitro of a nucleic acid, preferably a DNA encoding it.

According to this first aspect, the primer produced by means of a in vitro transcription may be synthesized from a DNA fragment encoding it, upstream which a transcription promoter site exists, for ex. the T7 promoter.

According to a second aspect, the RNA primer in the RNA primer/RNA template complex may be formed by in vitro transcripting a DNA insert inserted into an expression vector, a plasmid for instance, said vector further comprising at least the sequence of a transcription promoter that controls the DNA insert encoding the transfer RNA.

The RNA primer may also be formed by means of a chemical synthesis according to any method known to one skilled in the art. For example, chemically synthesizing a RNA primer included in a RNA primer/RNA template complex according to the invention may be conducted by means of methods described by Micura and al. (2002).

According to another aspect, the RNA primer may be a hemisynthetic molecule formed by coupling in order a plurality of RNA fragments constituting distinct parts of the RNA primer, each RNA fragment being either a in vitro transcription product of a DNA encoding said fragment, either the final product resulting from a chemical synthesis. Coupling the various successive RNA fragments in order so as to synthesize the RNA primer may be effected according to any method known to one skilled in the art. To conduct such a coupling between the RNA fragments, the man skilled in the art will advantageously refer to methods described by Zimmerman and al. and to references mentioned in the present application (Incorporation of Modified Nucleotides into RNA for studies on RNA Structure, Function and intermolecular Interactions (1998), Zimmerman R., Gait M J and Moore M J, Modification and Editing of RNAs, ASM Press, Washington D.C., 59-84).

RNA template included in the RNA primer/RNA template complex is advantageously formed by means of a in vitro transcription of a DNA encoding it, as previously defined in the present description.

DNA encoding viral RNA template is preferably inserted into an expression vector which further comprises at least one transcription promoter sequence that controls this DNA, as defined in example 1.

DNA encoding RNA template may advantageously be produced by cloning a DNA fragment formed by amplification, for ex. PCR amplification, of the DNA from an infectious viral clone, such as cloning PHXB2, described by Li and al. (1997).

The RNA template “codon sequence” (102) and primer binding sequence PBS (108) are advantageously adapted so as to be complementary to the transfer RNA “anticodon sequence” (101) and primer sequence (107), respectively, of the RNA primer/RNA template complex, so as to make sure that intermolecular helices (6C) and (7F) will be formed respectively in the RNA/RNA complex according to the invention.

Adapting the viral RNA template sequence to the RNA primer used to form the RNA primer/RNA template complex may be performed by means of a site-directed mutagenesis, as described for ex. by WAKEFIELD and al. (1994).

The RNA primer/RNA template complex is advantageously prepared according to the procedure described by ISEL and al. (1993).

Hybridization conditions used to form the RNA primer/RNA template complex will preferably be as follows:

RNA primer, for example a ligand-bearing RNA primer so as to subsequently maintain the primer onto the microplate surface, is incubated for 2 minutes at 90° C., for 2 minutes in ice, then for 20 minutes at 70° C. in NaCl 100 mM with an excess of RNA template, so as to ensure a complete hybridization of the primer.

According to another aspect, the RNA primer/RNA template complex may be formed at 37° C. with the nucleocapside protein NCp7 according to the method described by BRULE and al. (2002).

The RNA primer/RNA template complex according to the invention is brought into contact with the reverse transcriptase enzyme, preferably HIV-1 reverse transcriptase and with the inhibiting candidate compound to be tested in a known final concentration.

According to the method, step a) is conducted with an appropriate mixture of deoxyribonucleotide triphosphates (dNTPs) and dideoxyribonucleotide triphosphate (ddNTP), making possible to synthesize a polynucleotide which exclusively corresponds to the reverse transcription initiation step. The appropriate dNTPs/ddNTP mixture allows to elongate the primer sequence (107) of the RNA primer resulting in the synthesis of a sequence corresponding to the product +6 corresponding to the initiation.

For example, step a) is conducted with only three out the four natural deoxyribonucleotide triphosphates amongst A, T, G and C, the fourth nucleotide thus being a dideoxyribonucleotide triphosphate that is complementary to the nucleotide localized in position −6 as compared to the 3′ end of the primer binding sequence (108) on the RNA template. For example, in the case of the tRNA^{His} His[AC-ACG] complex illustrated in FIG. 1, step a) of the method is preferably conducted with the three deoxyribonucleotides T, G and C, dideoxyribonucleotide A being added thereto, which is complementary to ribonucleotide U localized on the sequence (118) in position −6 as compared to the 3′ end of the RNA primer binding sequence PBS (108). The same dideoxyribonucleotide A will be added to the three deoxyribonucleotides T, G and C when step a) is carried out with the adapted tRNA^{Met}/vRNA complex illustrated in FIG. 2, since the nucleotide localized in position −6 as compared to the 3′ end of the primer binding sequence PBS (108) is also the Uridine base.

In the above embodiment, an RNA primer elongation proceeds in the RNA primer/RNA template complex that specifically corresponds to the reverse transcription initiation step of the HIV-1 viral RNA.

Thus, the above screening method for compounds inhibiting the initiation of HIV-1 virus RNA reverse transcription is characterized in that step a) is conducted with an appropriate dNTPs and ddNTP mixture.

At least one of the natural deoxyribonucleotides or the dideoxyribonucleotide used is labelled by a detectable molecule.

The detectable molecule is preferably a radioactive molecule selected from the group consisting of ³[H], ¹⁴[C], ³²[P] and ³³[P].

The above screening method is preferably characterized in that the RNA primer/RNA template complex is immobilized onto a substrate.

To immobilize the RNA primer/RNA template complex onto a substrate, for ex. the well surface of an assay microplaque, at least one of the RNAs of the RNA/RNA complex comprises a ligand molecule that can specifically bind to a receptor molecule on the substrate surface.

In any case, the RNA primer 3′ end of the RNA primer/RNA template complex must remain free to function as a primer during the reverse transcription initiation. The ligand molecule that can specifically bind to the receptor molecule on the test substrate, for example the microplate well surface, may be localized on the RNA primer 5′ end or on one of the RNA template 5′ or 3′ ends of the RNA primer/RNA template complex.

According to another interesting aspect, RNA primer and/or RNA template comprises a ligand molecule that is chemically bound to any one of bases thereof, other than a 5′ or 3′ end base.

According to a preferred embodiment of the RNA primer/RNA template complex of the invention, the ligand molecule is a biotin. To introduce a biotinylated base into the RNA primer sequence of the RNA primer/RNA template complex of the invention, it will be possible to conduct the transcription of DNA encoding transfer RNA primer with a reduced ribonucleotide UTP concentration and with the modified ribonucleotide biotin-16-UTP.

Any other modification of RNA primer or RNA template forming the RNA primer/RNA template complex of the invention may be conducted according to methods known per se.

The man skilled in the art will advantageously refer to Hermanson's book published in 1996.

Most preferably, that is the RNA primer that comprises a ligand molecule.

Ligand/receptor molecule couples are preferably selected from the group consisting in:
biotin/streptavidin
biotin/avidin
biotin/RNA aptamer that recognizes biotin
fucose/avidin
RNA-His-tag/NiNTA
aptamer in primer 5′ end that recognizes streptavidin on a substrate
CoA or NAD or FAD on RNA/receptor 5′ end of these molecules (efficient incorporation of CoA, NAD and FAD into RNA by in vitro transcription, Huang F., 2003).

The ligand molecule is advantageously a biotin which can bind specifically to streptavidin. In that case, the immobilization of RNA/RNA biotin complex may be done on a substrate the surface of which has been previously covered with streptavidin molecules. According to this particular embodiment, the screening method for compounds inhibiting the initiation of HIV-1 reverse transcription according to the invention is characterized in that step a) is conducted in a reaction chamber, for instance the well of a test microplaque, the surface of which provides a substrate for immobilizing the RNA primer/RNA template complex.

The reaction chamber surface, for ex. a microplate well, is preferably covered with a receptor molecule that can bind specifically to a RNA/RNA complex-beared ligand molecule. An illustration thereof is given in the examples by fixing biotinylated RNA/RNA complex onto the microplate well surface that has previously covered with streptavidin.

To conduct the above screening method, the initiation activity of the reverse transcription is preferably quantified by measuring the radioactivity present in the polynucleotide which is synthesized by elongating RNA primer hybridized to RNA template within the RNA/RNA complex with the reverse transcriptase enzyme and dNTPs/ddNTP mixture.

According to an interesting aspect of the above screening method, measuring the radioactivity, which corresponds to the detection of extension products of tRNA primer, uses the “proximity scintillation” principle, that has been particularly described in the American patent U.S. Pat. No. 4,568,649. According to this principle, only the radioactive molecules that are located close to test substrate surface, which also comprises a scintillation radioactivity detection product, results in the production of a scintillation signal. Scintillation signal-inducing radioactive molecules are radioactive deoxyribonucleotides or dideoxyribonucleotides incorporated into RNA primer by elongating the primer sequence (107) that are located close to the test substrate surface, for example the microplate well surface. On the other hand, radioactive deoxyribonucleotides or dideoxyribonucleotides which have not been incorporated into transfer RNA by elongating the primer sequence (107) do not produce any detectable signal. This proximity scintillation principle on adapted test microplates has especially been used by EARNSHAW and POPE (2001), whose works can be advantageously considered as references for the one skilled in the art to put into practice the inhibitor screening method according to the invention.

According to a first preferred aspect for implementing the screening method for compounds inhibiting the HIV-1 virus reverse transcription according to the invention, the RNA primer/RNA template complex comprises RNA primer containing a sequence as set forth in SEQ ID NO 1 and a RNA template containing a sequence as set forth in SEQ ID NO 2, respectively.

According to a second preferred aspect for implementing the screening method for compounds inhibiting the HIV-1 virus reverse transcription according to the invention, the RNA primer/RNA template complex comprises RNA primer containing a sequence as set forth in SEQ ID NO 3 and a RNA template containing a sequence as set fort in SEQ ID NO 4, respectively.

The results obtained in the examples reveal that a reverse transcription nucleoside inhibitor such as AZTTP can inhibit HIV-1 virus reverse transcription initiation with an improved efficiency in the presence of increasing concentrations of this inhibition compound.

From the theoretical selectivity value of AZTTP incorporation as compared to dNTTP, the results obtained in the examples demonstrate that the resulting inhibition values of the reverse transcription initiation step are coherent with expected theoretical values and that the screening method such as defined hereabove makes it possible to detect the synthesis of a RNA primer elongation product corresponding to the HIV-1 reverse transcription initiation et that it can also measure initiation inhibition of HIV-1 reverse transcription.

Screening method for compounds inhibiting HIV-1 reverse transcription such as defined hereabove is preferably conducted with increasing concentrations of a candidate compound to be tested.

Comparison between the reverse transcription initiation activity level measured in step b) and the reverse transcription initiation activity level measured with no inhibiting candidate compound preferably consists in introducing in the same test a control sample comprising the RNA primer/RNA template complex, the reverse transcriptase enzyme and a mixture made of dNTPs and ddNTP similar to that used for the other test samples.

Alternatively, comparison made in step c) of the method is conducted against a known value of reverse transcription initiation activity level in a control sample without the candidate compound to be tested.

The screening method according to the invention advantageously further comprises positive control samples wherein known final concentrations of a known inhibitor, such as AZTTP, have been added to the RNA primer/RNA template complex of the invention+reverse transcriptase combination.

Moreover, to control that a candidate compound that has been selected as inhibiting compound according to the screening method hereabove has an exclusive and selective inhibition activity of the only initiation step of the HIV-1 virus reverse transcription, said method may further comprise following steps consisting in:

f) bringing into contact in a sample:
(i) a nucleotide primer that can mimic the elongation step and that is complementary to a viral RNA sequence, preferably complementary to the PBS sequence of the viral RNA;
(ii) a HIV-1 reverse transcriptase;
(iii) the dNTPs/ddNTP mixture used in step a) with the candidate compound tested in step a);

g) measuring the elongation activity of the nucleotide primer;

h) comparing the activity measured in step g) with the elongation activity measured in a control sample for which step f) has been conducted without the candidate compound;

i) determining the inhibition activity of the candidate compound on the basis of the activity comparison made in step h);

j) comparing the inhibition activity of the same candidate compound as determined in step e) with the inhibition activity of the same compound as determinated in step i).

Should the comparison in step i) reveal that the inhibition activities of the candidate compound, in step e) and in step i) respectively, are of the same order, said candidate compound may be categorized as a compound inhibiting the initiation step of HIV-1 virus reverse transcription, but non specific of this initiation step.

Should the comparison in step i) reveal that the inhibition activity of the candidate compound in step e) is higher than the inhibition activity measured in step i) or, alternatively, that the candidate compound does not possess any inhibition activity in step i), said candidate compound can be categorized as a compound specifically inhibiting the initiation step of the HIV-1 virus reverse transcription.

It is also an object of the present invention to use a RNA primer/RNA template complex such as defined hereabove in a screening method for compounds inhibiting the HIV-1 virus reverse transcription initiation.

It is also an object of the present invention to provide a kit for screening compounds inhibiting the HIV-1 virus reverse transcription initiation, characterized in that it comprises a RNA primer/RNA template complex such as defined hereabove.

According to a first aspect, the above screening kit is characterized in that it comprises a reverse transcriptase enzyme, preferably a HIV-1 reverse transcriptase enzyme.

According to a second aspect, the above screening kit is characterized in that it comprises an appropriate mixture of dNTPs and ddNTP making it possible to synthesize a polynucleotide produced by elongating the RNA primer during the reverse transcription initiation step.

Most preferably, at least one of the deoxyribonucleotides contained in the screening kit is labelled with a detectable molecule, preferably a radioactive molecule selected from the group consisting in ³[H], ¹⁴[C], ³²[P] and ³³[P].

The present invention will be further illustrated in the following non limitative examples.

### EXAMPLES

### Example 1

### In vitro Preparation of a Mimicking RNA Primer/RNA Template Complex and Structure of the Initiation Complex in the HIV-1 Reverse Transcription

A RNA primer/RNA template complex mimicking the initiation complex of the HIV-1 reverse transcription can be prepared in vitro from a transcription product of tRNA^{His} hybridized to its “adapted” vRNA (vRNA [His-AC-GAC]).

A. Material and Methods

A.1 Preparation of the RNA Primer

The plasmid containing the sequence encoding tRNA^{His}, pltRNA^{His}, was obtained by inserting into the EcoRI and Xmal sites of Puc18 a DNA fragment corresponding to tRNA^{His} sequence and possessing upstream the promoter site for the RNA polymerase of phage T7. This fragment was created by hybridizing and ligating appropriate oligodeoxyribonucleotides.

The transcription product of tRNA^{His} has been prepared by in vitro transcription of vector pltRNA^{His} previously digested by the Nsil restriction enzyme. The tRNA primer produced by in vitro transcription may bear a modification that makes it possible during the reverse transcription assay to maintain the elongated RNA primer/RNA template complex on a substrate bearing a ligand of this modification. The modification can be a biotin or any other ligand or reactant enabling the covalent or non-covalent anchorage for binding the complex to a solid substrate. There are a plurality of methods that can be considered to modify RNA (see for ex. Hermanson, 1996). Our example uses a biotinylated tRNA^{His} primer, biotinylation occurring during transcription with 40 mM Tris HCl, pH8 (37° C.), 15 mM MgCl₂, 50 mM NaCl, 1.6 mM spermidine, 50 U RNAsine, 5 mM DTE, 4 mM ATP, 4 mM CTP, 4 mM GTP, 2.5 mM UTP, 0.4 mM biotin-16-UTP; 22.5 U T7 RNA polymerase. For non biotinylated RNA synthesis, biotin-16-UTP is leaved out and UTP concentration is increased up to 4 mM. After transcription, plasmid DNA is digested with DNAse I and the transcription products are purified on HPLC columns.

A.2 Preparation of the RNA Template

Adapted vRNA, vRNA [His-AC-GAC], used in our trials corresponds to the 295 first nucleotides on the 5′ end of the isolation product HxB2 of HIV-1 and contains mutations enabling to form reverse transcription initiation characteristic structure (FIG. 1); PBS site is mutated to be complementary to the 18 nucleotides on the tRNA^{His} 3′ end; nucleotides of the A-rich region upstream of the PBS site are mutated so as to be complementary to the tRNA^{His} anticodon loop; other nucleotides in the vicinity of the PBS are mutated, thus increasing the complementarity to tRNA^{His} (Zhang and al., 1998). Plasmid plHis-AC-AGC containing sequences encoding adapted vRNA [His-AC-GAC] was constructed by inserting into the EcoRI and Xmal sites of Puc18 vector, from a DNA fragment produced by PCR from infectious molecular clone pHXB2(His-AC-AGC) (Li and al., 1997) provided by Dr. C. Morrow (Birmimgham, USA) and comprising T7 promoter upstream the reverse transcription initiation site. PCR primers have been prepared so as to amplify the region corresponding to the 732 nucleotides on the vRNA [His-AC-AGC] 5′ end.

The first 295 nucleotides of vRNA [His-AC-AGC] have been obtained by in vitro transcription of vector plHis-AC-AGC, previously digested by Rsal according to the procedure described hereabove and (Marquet and al., 1991). RNA has been purified as previously described.

A.3 Preparation of the HIV-1 RT

Plasmids used for producing HIV-1 RT, as well as the purification procedure have been provided by Dr. T. Unge.

A.4 S1 Nuclease Mapping of RNA Primer

In order to carry out experiments on solution enzyme mapping of RNA primer, either in a free state or hybridized to the template, tRNA^{His} has been labelled on its 5′ end. tRNA^{His} (15 μg) is incubated for 30 minutes at 37° C. in a CIP buffer (Calf Intestinal Phosphatase), with 15 U CIP. After phenol extraction and precipitation, tRNA is purified on a denaturant polyacrylamide gel. tRNA is then labelled on its 5′ end by transferring radioactive phosphate of [γ³²]ATP. tRNA (3 μg) is then incubated for one hour at 37° C. in 15 μl kinase buffer with 100 μCi [γ³²]ATP and 15 U phage T4 polynucleotide kinase, before being purified on a denaturant polyacrylamide gel.

The template/primer complex is prepared as described hereafter and according to procedures previously published (Isel and al., 1993). Alternatively, the complex formation may by carried out at 37° C. with nucleocapside protein NCp7 (Brulé and al., 2002).

tRNA^{His} mapping with or without vRNA [His-AC-AGC] by means of S1 nuclease is performed according to following procedure: 100000 cpm tRNA^{His} [γ³²]P-labelled on its 5′ end are incubated with or without 12 pmol vRNA [His-AC-AGC] for 2 minutes at 90° C., then for 2 minutes in ice. RNAs are then incubated for 20 minutes at 70° C. in 50 mM sodium cacodylate (pH 7.5), 300 mM KCl, then the complex is brought back to room temperature for 5 minutes in 50 mM sodium cacodylate (pH 7.5), 300 mM KCl, 5 mM MgCl₂, 1 mM ZnCl₂. Free tRNA^{His}, supplemented with 1 μg tRNAtotal or hybridized to RNA [His-AC-AGC] is then incubated with or without 200 U S1 nuclease for 7.5 or 15 minutes at 37° C. Reaction is stopped by S1 nuclease digestion with 20 μg proteinase K for 30 minutes at 37° C. After phenol extraction, RNAs are precipitated with ethanol, centrifugated and separated by 15% denaturant polyacrylamide gel electrophoresis.

B. Results

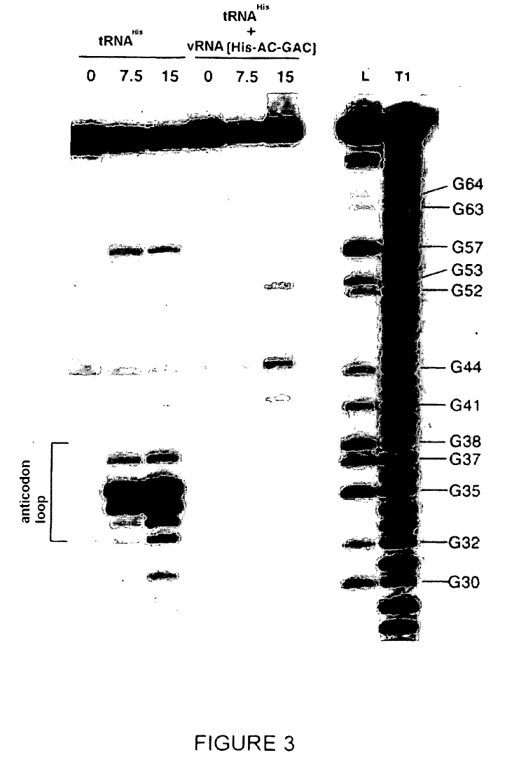

Surprisingly, these experiments reveal that the transcription product tRNA^{His} with no modified bases makes it possible to form a stable complex that mimics structural specificities of the reverse transcription initiation complex obtained with natural tRNA₃^{Lys} and its complementary vRNA. In particular, the anticodon loop of the transcription product tRNA^{His} that is reactive to S1 nuclease without the template is protected by vRNA [His-AC-GAC], thus implying an interaction with CCACAA loop of this RNA (FIGS. 1 and 3). Protection of the primer anticodon region against S1 nuclease represents one of the signatures of the initiation complex and one of the most important ways to control that template/primer interactions are not limited to the PBS region.

The template/primer complex prepared in vitro in which the primer anticodon region interacts with one RNA template region upstream of PBS site must be tested for its capacity to respond to reverse transcription initiation/elongation reaction criteria such defined by the authors (Isel and al., 1996; Lanchy and al., 1996; Lanchy and al., 1998).

### Example 2

### A vRNA [His-AC-GAC] Template/tRNA^{His} Primer Complex Possesses the Functional Characteristics of a Reverse Transcription Initiation Complex

In vitro reverse transcription assays according to procedures that have been already described (Isel and al., 1996) have reveal that the vRNA [His-AC-GAC]/tRNA^{His} transcripted complex also possesses kinetic characteristics of the HIV-1 initiation complex, that is to say a specific initiation step, followed by a non specific elongation step.

A. Material and Methods

In order to test in vitro reverse transcription, tRNA^{His} and ODN_{His} primers must be radio-labelled. tRNA^{His} is labelled according to the procedure described hereabove. ODN_{His} is labelled by transferring radioactive phosphate from [γ³²]ATP according to following procedure: 0.1 nmol ODN_{His} are incubated for one hour at 37° C. in a kinase buffer with 100 μCl [γ³²]ATP and 15 U polynucleotide kinase of phage T4 before being purified on denaturant polyacrylamide gel.

2 nM vRNA [His-AC-GAC] template/tRNA^{His} primer complex ([γ³²]P-labelled, hybridized to an excess of 2.5×vRNA [His-AC-GAC] according to the procedure described hereabove in 100 mM NaCl) or 19 nM vRNA [His-AC-GAC]/ODN_{His} complex ([γ³²]P-labelled, hybridized to an excess of 2.5×vRNA [His-AC-GAC] according to the procedure described hereabove in 100 mM NaCl) are pre-incubated for 4 minutes with ultimately 20 nM or 30 nM RT of HIV-1 in 50 mM Tris-HCl pH 7.5, 50 mM KCl, 6 mM MgCl₂ and 1 mM DTE, respectively. The reverse transcription reaction is initiated by adding 50 μM of each dNTP and stopped at different times, from 15 seconds to 30 minutes, with Tris-Borate 90 mM, pH 8.3, EDTA 25 mM. Reaction products are separated by 8% denaturant polyacrylamide gel electrophoresis.

B. Results

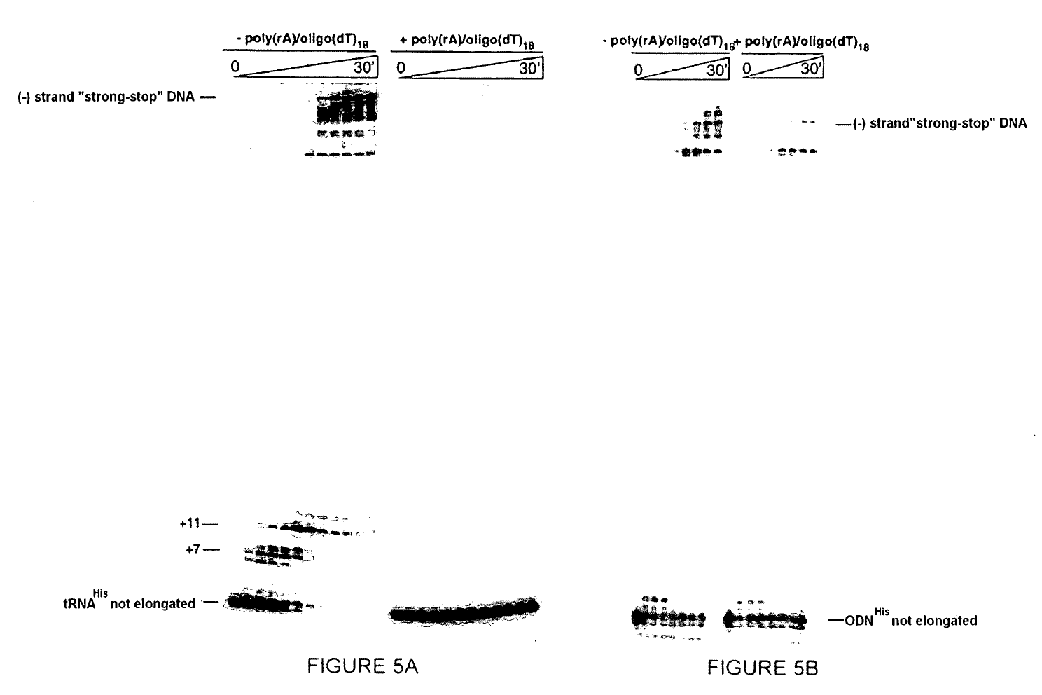

In the presence of tRNA^{His} primer, (−) strand strong-stop DNA synthesis proceeds in two steps (FIG. 5): addition of the first seven nucleotides to the tRNA^{His} primer occurs in a relatively non processive way as shown by intermediates which accumulate between positions +1 and +7 (FIG. 5), but which disappear with the time. This step corresponds to the initiation stage as previously defined for the natural system (Isel and al., 1996; Lanchy and al., 1996; Lanchy and al., 1998). It is followed by an elongation step, more processive, wherein “pauses” are not so numerous. As for the natural system, the elongation step may be mimiced by an oligodeoxyribonucleotide primer complementary to the PBS (ODN_{His}) which enables a processive reverse transcription, as is shown by the lack of “pauses” (FIG. 5).

The low processivity of reverse transcriptase during initiation with the tRNA^{His} primer is confirmed by the lack of DNA synthesis when a poly(rA)/(dT)₁₈ trap is provided (FIG. 5). This result is similar to that obtained with the natural primer tRNA₃^{Lys}. By contrast, when ODN_{His} primer is used, (−) strand strong-stop DNA synthesis remains detectable with poly(rA)/oligo(dT)₁₈ trap, indicating that as in the natural case, RT elongation is highly processive.

Initiation complex functional study, as compared to (−) strand strong-stop DNA synthesis profiles that have been obtained with a tRNA primer or with an oligodeoxyribonucleotide primer represents the functional means of control that enables to validate a template/primer complex as miming the reverse transcription natural complex.

### Example 3

### Initiation as Target in a High-Throughput Screening Test

Template/primer complexes described hereabove that mimic the specific reverse transcription initiation complex may be used in a screening test with high throughout of reverse transcription inhibitors specifically targeting this initiation step.

A. Material and Methods

In the context of the screening test, the biotinylated vRNA [His-AC-GAC] template/tRNA_{His} primer complex is preformed according to the procedure described hereabove and (Isel and al., 1993). Alternatively, the complex formation can be conducted at 37° C. with nucleocapside protein NCp7 (Brulé and al., 2002). The complex in an appropriate reverse transcription buffer (Tris-HCl 50 mM, pH 7.5; KCl 50 mM; MgCl₂ 6 mM; DTE 1 mM and (Isel and al., 1996)) is then transferred into the wells of a microplate (for ex. “Flashplate”, NEN).

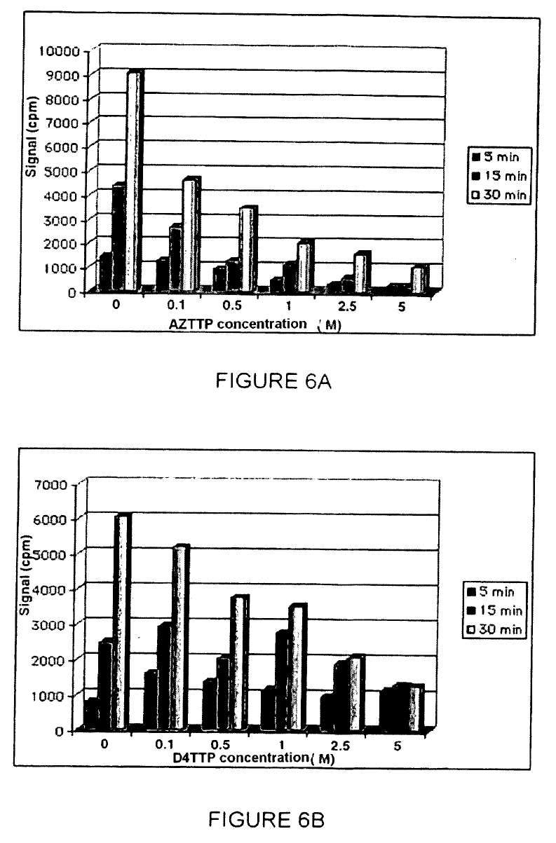

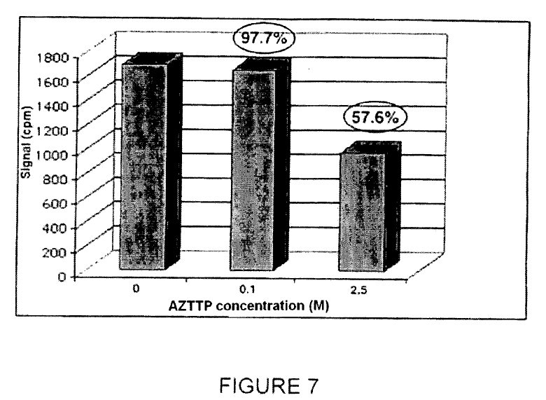

In order to detect a (−) strand strong-stop DNA synthesis, 10 nM template/primer complex are elongated by HIV-1 reverse transcriptase, of wild-type or mutated in its Rnase H site, with 5 μM of each dNTP (dGTP, dCTP, dATP and dTTP/dTTP[³H] (specific activity: 121 Ci/mmol; Amersham)), with or without reverse transcription inhibitors (AZTTP or d4TTP in the examples of FIG. 6), for 5, 15 and 30 minutes at 37° C. Reactions are stopped with EDTA 25 mM and detection of extension products proceeds according to the “proximity scintillation” principle or SPA (scintillation proximity assay) protected by a patent filed by Amersham (U.S. Pat. No. 4,568,649). In this system, any not bound radioelement that is to say not incorporated into the primer, is not detected and only a target-bound radioelement will produce a scintillation signal. The signal is proportional to the amount of extension products. Proximity scintillation principle on a “flashplate” has been widely used in this application type (Earnshaw and Pope, 2001). Bound radioactivity onto the surface of each microplate well is counted with a MicroBeta counter (Wallac-Perkin-Elmer).

These experiments demonstrate that it is possible in this formalism to detect the (−) strand strong-stop DNA synthesis as well as inhibition thereof with AZTTP and d4TTP (FIG. 6). (−) strand strong-stop DNA synthesis inhibition is comparable to that obtained in a solution in different test systems. With 5 μM AZTTP, the inhibition rate is about 90% after a 30 minute-reaction. In accordance with results published in the past (Arts and al., 1996a; Isel and al., 2001), the inhibition rate with the same concentration of d4TTP is lower (80%).

To detect the synthesis of a product corresponding to the reverse transcription initiation, that is to say to the addition of the first 6 nucleotides to biotinylated tRNA^{His} primer, 30 nM of template/primer complex are elongated with 90 nM HIV-1 RT of the wild-type or mutated in its Rnase H site with dGTP, dCTP, dTTP/dTTP[³H] (specific activity: 121 Ci/mmol; Amersham), (5 μM each ultimately) and ddATP (20 μM), complementary to the 6^{th} nucleotide upstream of the PBS site, with or without 0.1 or 2.5 μM AZTTP (FIG. 7) (or molecules coming from data banks in posterior application of this test) for 20 minutes at 37° C. Reactions are stopped by adding EDTA 25 mM and extension product detection is performed as previously described.

B. Results

These experiments demonstrate that the synthesis of a product corresponding to reverse transcription initiation can be detected and, even more crucial, that a decreased signal in the presence of AZTTP is significant. For a AZTTP concentration of 0.1 μM, with an excess of 50×dTTP, experiment signal represents 92.3% (mean calculated on 3 tests) (Table I) of the signal obtained without any inhibitor. With 2.5 μM AZTTP and an excess of 2×dTTP, the signal represents 61% (mean calculated on 7 tests) (Table I) of the signal obtained without any inhibitor.

Based on the AZTTP incorporation theoretical selectivity against dTTP, a theoretical signal expected for a given AZTTP and dTTP ratio may be calculated and compared to the test signal. For a 1:2 AZTTP/dTTP ratio, expected theoretical signals for 0.6 and 0.7 selectivity values (selectivity measured in initiation and elongation (Rigourd and al., 2000)) are 60.7% and 56.7%, respectively. A mean made on 7 tests carried out under these conditions (AZTTP/dTTP=1:2) results in an test signal of 61±6% in concordance with the theoretical signal.

The concordance between theoretical signals (Table I) and test signals (FIG. 7), even with low AZT concentration, is positive and confirms the use of this system to screen initiation.

TABLE I
Comparison between expected theoretical signals and test signals
during the synthesis of a product corresponding to the reverse
transcription initiation. The expected theoretical signal is
calculated according to the AZTTP and dTTP concentration
ratio in the reaction medium and according to the AZTTP
incorporation selectivity against dTTP (as measured in initiation
and elongation (Rigourd and al., 2000)).

| | Theoretical signal (%) | | Test signal (%) |
|---|---|---|---|
| [dTTP]/AZTTP] = 2 | selectivity | 60.7 | 61 ± 6 |
| | (AZTTP/dTTP) = 0.6 | | |
| | selectivity | 56.7 | |
| | (AZTTP/dTTP) = 0.7 | | |
| [dTTP]/AZTTP] = 50 | selectivity | 97.7 | 92.3 ± 3.4 |
| | (AZTTP/dTTP) = 0.6 | | |
| | selectivity | 97.3 | |
| | (AZTTP/dTTP) = 0.7 | | |

### REFERENCES

BRULE f; and al., 2002, RNA, Vol. 8 (1):8-15

EARNSHAW D. and POPE A., 2001, J. Biomo Screen, Vol. 6:39-46

HERMANSON G., 1996, Bioconjuguate Techniques, Academic Press Inc., San Diego, USA

HUANG F., 2003, Nucleic Acids Res., Vol.31, DOI:10.1093/nar/gng008

HUANG Y., A SHALOM, Z. LI, J. WANG, J. MAK, M. A. WAINBERG and L. KLEIMAN, 1996. Effects of modifying the RNA₃^{Lys} anticodon on the initiation of human immunodeficiency virus type 1 reverse transcription, J. Virol. 70:4700-4706

ISEL C. and al., 1993, The Journal of Biological Chemistry, Vol. 268(34):25.269-25.272

ISEL C. and al., 1995, J. Mol. Biol., Vol. 1. 247:236-250

KANGS S-M and al., 1996, Virology, Vol. 222:401-414

KANGS S-M, Z. J. ZHANG and C. D. MORROW, 1997. Identification of a sequence within U5 required for human immunodeficiency virus type 1 to stably maintain a primer binding site complementary to tRNA^{Met}, J. Virol. 71:207-217

WAKEFIELD J. K., S. M. KANG and C. D. MORROW, 1996. Construction of a type 1 human immunodeficiency virus that maintains a primer binding site complementary to tRNA^{His}, J. Virol. 70:966-975)

LANCHY J. M. and al., 1990, The Embo Journal, Vol. 15, (24):7178-7187

LANCHY J. M. and al., 1998, J. Biol. Chem., Vol. 273, 24.425-24.432

LI Y and al., 1997, J. Virol. Vol. 71:6315-6322

LIANG C., X. LI, L. RONG, P. INOUYE, Y. QUAN, L. KLEIMAN and M. A. WAINBERG, 1997. The importance of the A-rich loop in human immunodeficiency virus type 1 reverse transcription and infectivity, J. Virol. 71:5750-5757

MARQUET R. and al., 1991, Nucleic Acids Research, Vol. 19(9):2349-2357

MICURA and al. 2002, Angew. Chem. Int. Ed. Engl., Vol. 41 (13): 2265-2269

SKRIPKIN E. and al., 1996, Nucleic Acids Research, Vol. 24:509-514

ZHANG Z., S. M. KANG, A. LEBLANC, S. L. HAJDUK and C. D. MORROW, 1996. Nucleotide sequences within the U5 region of the viral RNA genome are the major determinants for an human immunodeficiency virus type 1 to maintain a primer binding site complementary to tRNA^{His}, Virology, 226:306-317

ZHANG Z., S. M. KANG, Y. LI and C. D. MORROW, 1998. Genetic analysis of the U5-PBS of a novel HIV-1 reveals multiple interactions between the tRNA and RNA genome required for initiation of reverse transcription RNA. 4:394-406

ZIMMERMAN and al. Incorporation of Modified Nucleotides into RNA for studies on RNA Structure, Function and Intermolecular Interactions (1998)

ZIMMERMAN R., GAIT M. J. and MOORE M. J. Modification and Editing of RNAs ASM Press, Washington D.C., 59-84

WAKEFIELD J. K. and al., 1994; J. Virol., Vol. 68:1605-1614.

1.-24. (canceled)

25. An in vitro method for screening compounds inhibiting the initiation of HIV-1 virus RNA reverse transcription, comprising:
a) contacting in a sample:
(i) an RNA primer/RNA template complex formed between:
(1) an RNA primer comprising from the 5′ end to the 3′ end:
an intramolecular pairing sequence (109);
a single-strand sequence (111);
a stem-loop forming sequence (112);
a single-strand sequence (113);
an intermolecular pairing sequence (101) called anticodon sequence;
an intermolecular pairing sequence (103);
an intermolecular pairing sequence (105);
an intramolecular pairing sequence (110);
a single-strand sequence (114);
an intermolecular pairing sequence (107) used as a primer; and
(2) an RNA template comprising from the 5′ end to the 3′ end:
an intramolecular pairing sequence (115);
an intramolecular pairing sequence (116);
an intermolecular pairing sequence (106);
an intramolecular stem-loop forming sequence (117);
an intramolecular pairing sequence (104);
an intramolecular pairing sequence (102) called codon sequence;
an intramolecular pairing sequence (118);
a single-strand sequence (119);
an intermolecular pairing sequence (108);
an intramolecular stem-loop (8) forming sequence (120);
a single-strand sequence (121);
an intramolecular pairing sequence (122);
wherein the RNA primer consists of:
(a) either the in vitro transcription product of a nucleic acid encoding said RNA primer,
(b) or the chemical synthesis or chemical hemisynthesis product of said RNA primer; and RNA primer being chosen amongst RNA primer sequence SEQ ID NO1 and RNA primer sequence SEQ ID NO3,
being specified that:
the paired sequences (101) and (102) form a helix (6C);
the paired sequences (107) and (108) form a helix (7F);
the paired sequences (109) and (110) form a helix (A);
the paired sequences (107) and (108) form a helix (7F);
the sequence (112) forms a stem-loop (B);
the paired sequences (115) and (122) form a helix (1);
the paired sequences (116) and (118) form a helix (2);
the sequence (117) forms a stem-loop (4);
the sequence (120) forms a stem-loop (8);
with:
(ii) a HIV-1 reverse transcriptase enzyme; and
(iii) an appropriate dNTPs/ddNTP mixture;
(iv) in the presence of a candidate compound to be tested;
b) measuring the reverse transcription initiation activity in said sample;
c) comparing the measured activity in step b) to the reverse transcription initiation activity measured in a control sample for which step a) is conducted without said candidate compound.

26. The method of claim 25, wherein RNA primer represents the in vitro transcription product of a nucleic acid encoding it.

27. The method of claim 25, wherein RNA primer is obtained by means of a chemical synthesis.

28. The method of claim 25, wherein RNA primer is a hemisynthetic molecule formed by coupling in order a plurality of RNA fragments constituting distinct parts of the RNA primer, each RNA fragment being either an in vitro transcription product of a DNA encoding said fragment, either the final product resulting from a chemical synthesis.

29. The method of claim 25, further comprising:
d) selecting the candidate compound for which reverse transcription initiation activity measured in step b) is lower than that measured for the control sample; and
e) calculating the candidate compound inhibition activity selected in step d).

30. The method of claim 25, wherein in step a), the appropriate dNTPs/ddNTP mixture comprises three out the four natural deoxyribonucleotide triphosphates amongst A, T, G and C, the fourth nucleotide thus being a dideoxyribonucleotide triphosphate that is complementary to the nucleotide localized in position −6 as compared to the 3′ end of the primer binding sequence (108) on the RNA template.

31. The method of claim 25, wherein at least one of the natural deoxyribonucleotides or the dideoxyribonucleotide used is labelled by a detectable molecule.

32. The method of claim 31, wherein the detectable molecule is preferably a radioactive molecule further defined as ³[H], ¹⁴[C], ³²[P] or ³³[P].

33. The method of claim 32, wherein the initiation activity of the reverse transcription is quantified by measuring the radioactivity present in the polynucleotide which is synthesized by elongating RNA primer hybridized to RNA template within the RNA/RNA complex.

34. The method of claim 25, wherein the RNA/RNA complex is immobilized on a substrate.

35. The method of claim 34, wherein at least one of the RNAs in the RNA primer/RNA template complex comprises a ligand molecule that can specifically bind to a receptor molecule on the substrate surface.

36. The method of claim 34, wherein the ligand molecule is a biotin.

37. The method of claim 34, wherein step a) is conducted in a reaction chamber, the surface of which provides a substrate for immobilizing the RNA/RNA complex.

38. The method of claim 36, wherein the reaction chamber surface is covered with a receptor molecule that can bind specifically to an RNA primer/RNA template complex-borne ligand molecule.

39. The method of claim 25, wherein the RNA primer/RNA template complex comprises RNA primer containing a sequence as set forth in SEQ ID NO:1 and an RNA template containing a sequence as set forth in SEQ ID NO:2, respectively.

40. The method of claim 25, wherein the RNA primer/RNA template complex comprises RNA primer containing a sequence as set forth in SEQ ID NO:3 and an RNA template containing a sequence as set forth in SEQ ID NO:4, respectively.

41. The method of claim 25, further comprising:
f) bringing into contact in a sample:
(i) a nucleotide primer that can mimic the elongation step;
(ii) an HIV-1 reverse transcriptase;
(iii) the dNTPs/ddNTP mixture used in step a) with the candidate compound tested in step a);
g) measuring the elongation activity of the nucleotide primer;
h) comparing the activity measured in step g) with the elongation activity measured in a control sample for which step f) has been conducted without the candidate compound;
i) determining the inhibition activity of the candidate compound on the basis of the activity comparison made in step h);
j) comparing the inhibition activity of the same candidate compound as determined in step e) with the inhibition activity of the same compound as determined in step i).

42. An RNA primer/RNA template complex formed between:
(i) an RNA primer comprising from the 5′ end to the 3′ end:
an intramolecular pairing sequence (109);
a single-strand sequence (111);
a stem-loop forming sequence (112);
a single-strand sequence (113);
an intermolecular pairing sequence (101) called anticodon sequence;
an intermolecular pairing sequence (103);
an intermolecular pairing sequence (105);
an intramolecular pairing sequence (110);
a single-strand sequence (114);
an intermolecular pairing sequence (107) used as a primer; and
(ii) an RNA template comprising from the 5′ end to the 3′ end:
an intramolecular pairing sequence (115);
an intramolecular pairing sequence (116);
an intermolecular pairing sequence (106);
an intramolecular stem-loop forming sequence (117);
an intramolecular pairing sequence (104);
an intramolecular pairing sequence (102) called codon sequence;
an intramolecular pairing sequence (118);
a single-strand sequence (119);
an intermolecular pairing sequence (108);
an intramolecular stem-loop (8) forming sequence (120);
a single-strand sequence (121);
an intramolecular pairing sequence (122);
wherein the RNA primer consists of:
(c) either the in vitro transcription product of a nucleic acid encoding said RNA primer,
(d) or the chemical synthesis or chemical hemisynthesis product of said RNA primer; and RNA primer being chosen amongst RNA primer sequence SEQ ID NO:1 and RNA primer sequence SEQ ID NO:3,
being specified that:
the paired sequences (101) and (102) form a helix (6C);
the paired sequences (107) and (108) form a helix (7F);
the paired sequences (109) and (110) form a helix (A);
the paired sequences (107) and (108) form a helix (7F);
the sequence (112) forms a stem-loop (B);
the paired sequences (115) and (122) form a helix (1);
the paired sequences (116) and (118) form a helix (2);
the sequence (117) forms a stem-loop (4);
the sequence (120) forms a stem-loop (8).

43. The RNA primer/RNA template complex of claim 42, wherein at least one of the RNAs in the RNA/RNA complex comprises a ligand molecule that can specifically bind to a receptor molecule.

44. The RNA primer/RNA template complex of claim 43, wherein the ligand molecule is a biotin.

45. The RNA primer/RNA template complex of claim 42, further comprising an RNA primer containing a sequence as set forth in SEQ ID NO:1 and an RNA template containing a sequence as set forth in SEQ ID NO:2, respectively.

46. The RNA primer/RNA template complex of claim 42, further comprising an RNA primer containing a sequence as set forth in SEQ ID NO:3 and an RNA template containing a sequence as set forth in SEQ ID NO:4, respectively.

47. A method comprising using an RNA primer/RNA template complex of claim 42 in a screening method for compounds inhibiting the initiation of HIV-1 virus RNA reverse transcription.

48. A kit for screening compounds inhibiting the initiation of HIV-1 virus RNA reverse transcription comprising an RNA primer/RNA template complex of claim 42.
